(19) United States
(12) Patent Application Publication
(10) Pub. No.: US 2018/0050575 A1
(43) Pub. Date: Feb. 22, 2018

(54) Vehicle Occupant Detection System

(71) Applicants: Seth Campbell, Washington, UT (US); Richard Curtis Nordgran, Springville, UT (US); Weston Brent Johnson, St. George, UT (US); Gian Franco Sacco, Pasadena, CA (US); Paolo Focardi, Pasadena, CA (US)

(72) Inventors: Seth Campbell, Washington, UT (US); Richard Curtis Nordgran, Springville, UT (US); Weston Brent Johnson, St. George, UT (US); Gian Franco Sacco, Pasadena, CA (US); Paolo Focardi, Pasadena, CA (US)

(21) Appl. No.: 15/682,517

(22) Filed: Aug. 21, 2017

Related U.S. Application Data
(60) Provisional application No. 62/377,414, filed on Aug. 19, 2016.
(60) Provisional application No. 62/385,281, filed on Sep. 9, 2016.
(60) Provisional application No. 62/520,258, filed on Jun. 15, 2017.

(51) Int. Cl. B60H 1/00; B60R 21/015; G06K 9/00; G01N 33/00; G01S 7/52

(52) U.S. Cl. CPC B60H 1/00742; B60R 21/01536; G01S 7/52006; G06K 9/00832; G01N 33/004; B60H 1/00735

(57) ABSTRACT

A living vehicle occupant detection system has a control unit that is mounted within a vehicle, a temperature sensor which is mounted within the interior passenger compartment of the vehicle and is communicatively coupled (either wired or wirelessly) to the control unit, and a life detection unit that will indicate the presence and location of a heartbeat within the interior of the vehicle and that is communicatively coupled (either wired or wirelessly) to the control unit. A carbon monoxide (CO) sensor may also be installed within the interior of the vehicle to optimize CO detection and is coupled (either wired or wirelessly) to the control unit.

### CROSS-REFERENCE TO RELATED APPLICATIONS

This application claims the benefit of U.S. Provisional Application Ser. No. 62/377,414, filed on Aug. 19, 2016, and U.S. Provisional Application Ser. No. 62/385,281, filed on Sep. 9, 2016, and U.S. Provisional Application Ser. No. 62/520,258, filed on Jun. 15, 2017, all of which are incorporated herein by reference in their entireties.

### TECHNICAL FIELD

The present disclosure relates to vehicle systems and occupant safety. More specifically, the present disclosure relates to systems and methods for detecting living occupants within a vehicle and for initiating one or more countermeasures to prevent, or reverse, dangerous conditions.

### BACKGROUND

As is well-known, the internal temperature of a parked vehicle will vary in comparison to the ambient temperature on the outside of the vehicle. This is due in large part to the greenhouse effect, which generally causes the internal temperature of a vehicle to exceed the external temperature. For example, on a sunny day, the interior temperature of a vehicle can rise 30 degrees or more above the ambient temperature in about 20 minutes, with minimally lowering or “cracking” of the window having almost no effect on the rate of temperature increase. Due to this marked increase in temperature, the conditions for occupants inside of a parked, non-running vehicle quickly become dangerous, resulting in a number of deaths each year. These deaths include both people and animals, and, in large part, are accidental.

As such, a number of systems have been invented in the prior art in an attempt to save lives. Systems range from parent/caregiver notification, to vehicle automation (e.g., starting the vehicle and A/C components), etc. However, one of the main difficulties in the art has been the determination of when an occupant has been left in a vehicle so that the above-mentioned countermeasures may be initiated. Additionally, not all counter-measures are appropriate. For example, starting the vehicle may create additional unsafe conditions, such as carbon monoxide, if the vehicle is inside of a garage or has a defective emissions system.

Additionally, some systems in the prior art leave the vehicle vulnerable to tampering, which may create a false-positive, allowing an intruder to access the vehicle. For example, systems in the prior art incorporate the rolling-down of windows when temperature thresholds are exceeded. If a false positive can be easily triggered by an intruder, the vehicle, and/or its contents, is susceptible to theft.

Therefore, there is a need for a system that can accurately detect a living occupant, can monitor the conditions inside of the car, and that can take remedial steps so as to reduce the risk of death to the occupant. The present invention seeks to solve these and other problems.

### SUMMARY OF EXAMPLE EMBODIMENTS

In one embodiment, a vehicle occupant detection system comprises a life detection unit for detecting the presence of a living occupant and an alerting component to alert the driver and/or others if the interior temperature of the vehicle has reached a predetermined level, so that corrective action can be taken to prevent harm to the vehicle occupant(s). In one embodiment, the life detection unit comprises a microwave transmitter, a microwave receiver, and a wireless transceiver. In one embodiment, the alerting component comprises a microcontroller configured to activate the alerting means at a triggering event.

In one embodiment, a vehicle occupant detection system comprises a control unit that is mounted within a vehicle, a temperature sensor which is mounted within the interior passenger compartment of the vehicle and is communicatively coupled (either wired or wirelessly) to the control unit, and a life detection unit that will indicate the presence and location of a live occupant within the interior of the vehicle and that is communicatively coupled (either wired or wirelessly) to the control unit. In one embodiment, a carbon monoxide (CO) monitor is installed within the interior of the vehicle to optimize CO detection and is coupled (either wired or wirelessly) to the control unit.

### BRIEF DESCRIPTION OF THE DRAWINGS

FIG. 1 is a flowchart illustrating a vehicle occupant detection system;

FIG. 2 is a flowchart illustrating a vehicle occupant detection system; and

FIG. 3 is a flowchart illustrating a vehicle occupant detection system.

### DETAILED DESCRIPTION OF EXAMPLE EMBODIMENTS

The following descriptions depict only example embodiments and are not to be considered limiting of its scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various different sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

As used herein, “occupant” or “living occupant” is used to describe a person or animal with a heartbeat.

As disclosed herein, a vehicle occupant detection system reduces the risk that an occupant will perish while remaining in a parked vehicle by detecting the movement, the heartbeat, or the breathing of an occupant and initiating one or more steps to mitigate hazardous conditions. As will be appreciated from the below disclosure, some benefits of using heartbeat and breathing detection to detect occupants include: 1) the ability to detect an occupant regardless of size, weight, or position in the car; and, 2) the ability to detect not only humans, but animals as well. As such, it will be appreciated that the vehicle occupant detection system disclosed herein solves many important needs in the art.

In one embodiment, a vehicle occupant detection system comprises a life detection unit for detecting the presence of a living occupant and an alerting component to alert the driver and/or others if the interior temperature of the vehicle has reached a predetermined level, so that corrective action can be taken to prevent harm to the vehicle occupant(s). The life detection unit comprises a microwave transmitter and receiver, wherein the microwave transmitter transmits a signal, with the reflected signals being received by the receiver. The vehicle occupant detection system then processes the reflected signals (e.g., using a processor, microcontroller, or similar) and is configured to determine the presence of an occupant based upon bodily movement, including external movements or internal movements, such as a heartbeat, lung movement, etc. In other words, as microwave signals are reflected and received, it is possible to detect the presence of an occupant by comparing the reflected signals. This is due to the fact that an occupant will have one or both of outward movement (e.g., moving arms and legs) and inward movement (e.g., heartbeat, lungs, etc.). As the signal is reflected off of an occupant (“occupant signals”), the occupant signals have variations due to the movement of the body, either externally or internally (the occupant signals are dynamic), when compared against each other, deducing that an occupant is present. If no occupant is present, the signals received remain static (no movement), indicating no life within the vehicle. Similar technologies may be used that are present in the art, such as those disclosed in Publication WO2016025961 filed on Aug. 17, 2015 by Lux, Publication WO2009083017 filed Dec. 27, 2007 by Borlez, and U.S. Publication US20130001422 filed Jun. 28, 2012 by Lavon, each disclosure being incorporated herein by reference in their entireties. The alerting component may comprise triggering the alarm system of the vehicle, the horn, or may contact the owner of the vehicle directly utilizing cellular networks and similar.

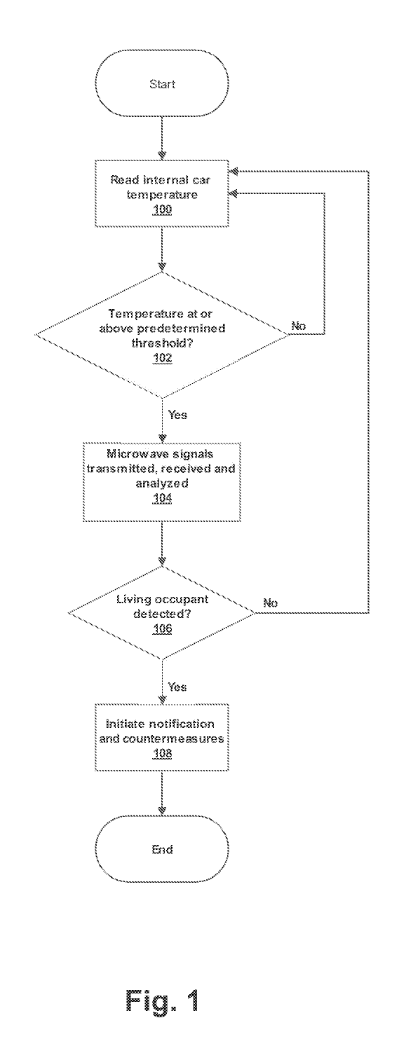

FIG. 1 shows a flowchart for using a vehicle occupant detection system to detect the presence of a living occupant within a vehicle. In one embodiment, a temperature sensor is mounted within the interior passenger compartment of the vehicle. Accordingly, at step 100, the internal temperature is measured. At 102, if the temperature does not exceed a predetermined threshold (e.g., 90 degrees Fahrenheit), the system loops. If the temperature does exceed the predetermined threshold, then at step 104 a microwave transmitter begins transmitting signals, which are then reflected from within the vehicle and received by a receiver. At 106, a comparator, microcontroller, or other processor compares the received signals. If the received signals are all static (i.e., no variation between received reflected signals), then no living occupant is detected. The system loops once again. However, if the received signals are not static (i.e., the received microwave signals have variations (occupant signals)), this is indicative of a living occupant, and at step 108, notifications and countermeasures are initiated. A non-limiting example of a notification would be a phone call and/or text message to the owner/operator's cell phone and an example countermeasure would be to start the vehicle and activate the HVAC system. It will be appreciated that while temperature sensor is described above for measuring temperature, a heat index sensor may also be utilized, which measures both temperature and humidity. It is known that prolonged exposure to a heat index of 90 degrees Fahrenheit or higher is dangerous. However, while 90 degrees Fahrenheit is described herein, the invention is not so limited, and the temperatures may fluctuate according to location and use. Further, while heat is used as an example, plummeting temperatures may also be harmful. Accordingly, in cold temperature settings, the vehicle may be started along with the heat of the HVAC system.

In one embodiment, a vehicle occupant detection system comprises a control unit that is mounted within a vehicle, a temperature sensor which is mounted within the interior passenger compartment of the vehicle and is communicatively coupled (either wired or wirelessly) to the control unit, and a life detection unit that will indicate the presence of an occupant within the interior of the vehicle and that is communicatively coupled (either wired or wirelessly) to the control unit. The control unit comprises a microcontroller or other logic control means. In one embodiment, the life detection unit comprises a microwave transmitter and microwave receiver. Therefore, in one example of use, the control unit is configured to activate the life detection unit when the temperature sensor reaches a predetermined threshold. The life detection unit then emits signals which are received and then analyzed by the control unit. If the received signals are indicative of a living occupant (e.g., received signals vary from one to the next), then countermeasures and/or alerts may be initiated by the control unit. Alerts may be in the form of audible sounds at the vehicle (e.g., car alarm, horn, etc.), phone calls, text messages, or other alerts. Countermeasures may include rolling down the windows, starting the vehicle and its HVAC system, or other measures. These alerts and countermeasures are easily accomplished by the control unit being in communication with vehicle computer components, ignition circuits, and other known mechanisms for controlling the vehicle. For example, many vehicles today utilize remote engine start with auto HVAC initiating. Accordingly, the control unit may be communicatively coupled with the receivers, controllers, or other vehicle components to initiate these same controls. Further, the control unit, in one embodiment, may be equipped with components for remotely starting the vehicle, should the vehicle not be equipped with such capability. In such a scenario, remote start components that are known in the industry are incorporated into the control unit. In one embodiment, a carbon monoxide (CO) monitor is installed within the interior of the vehicle to optimize CO detection and is coupled (either wired or wirelessly) to the control unit. For example, if the vehicle is within an enclosed space (e.g., home garage) when the vehicle is started as a countermeasure (i.e., if an occupant is detected and temperature thresholds are exceeded), it is important to ensure that CO levels do not become harmful within the vehicle. In the event CO levels exceed a predetermined threshold (e.g., 35 ppm), the vehicle may be turned off again while escalating alerting means (e.g., additional notifications to owner/operator, car alarms, audible voice alerting those nearby to break windows, etc.).

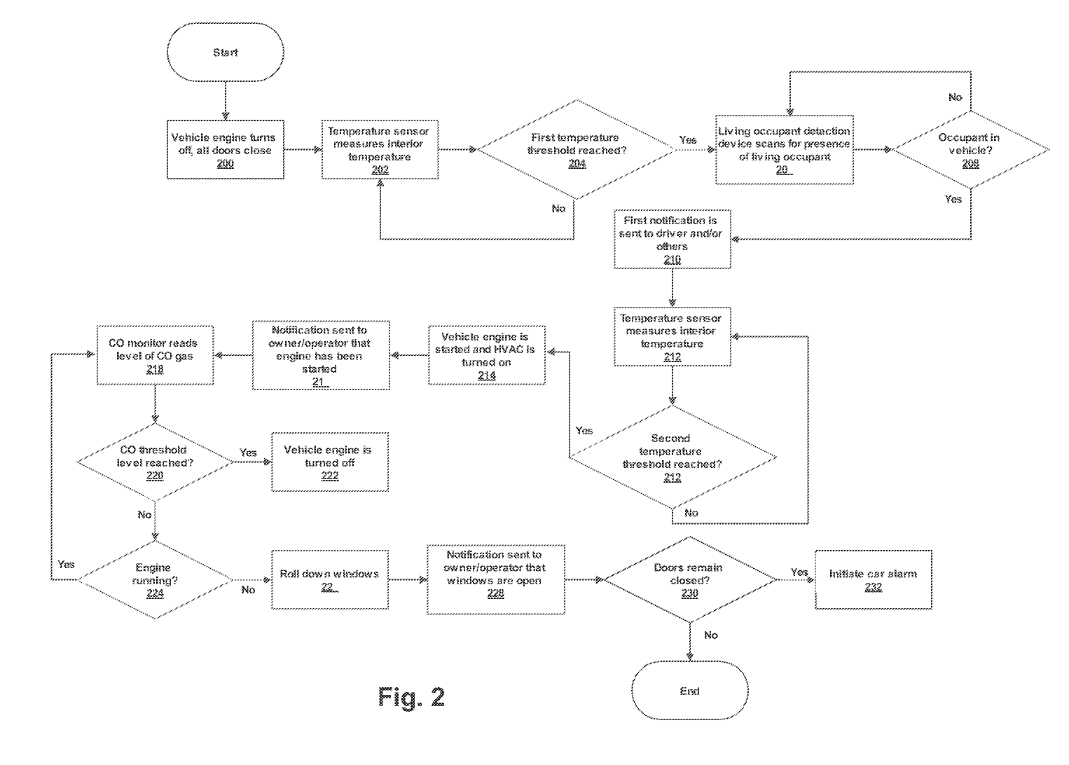

In one embodiment, as illustrated by the flow chart in FIG. 2, the control unit of a vehicle occupant detection system initiates at 200 when it detects that the engine of the vehicle has been shut off and all doors are closed. In other words, the control unit is directly connected to a power source (e.g., vehicle battery) so as to remain functional despite the engine and alternator not running. Battery charge monitoring systems known in the art may be incorporated so as to ensure that the control unit will not drain the battery beyond its ability to start the vehicle. With the doors closed, the temperature sensor at 202 measures the temperature of the interior of the vehicle and will continue to measure the interior temperature until the system is terminated. If a first predetermined temperature threshold is reached at step 204, then at step 206 the life detection unit scans (i.e., transmits and receives microwave (or equivalent) signals) for a heartbeat (or other movement) in the interior of the vehicle to detect a living occupant in the vehicle. The distance and penetration of the transmitted microwave signals are controlled by the frequency of the microwaves. For example, using a lower frequency, such as 2.5 GHz, allows the transmitted microwaves to travel longer distances and penetrate more surfaces. On the other hand, a higher frequency such as 66 GHz keeps the signal within the vehicle or at its immediate surroundings, which may also depend upon the antenna patterns. If the radio transmitter is mounted to the ceiling of the vehicle, the signals are then transmitted downward and would be deflected at the pavement, reducing interference and false positives from neighboring vehicles or persons. However, ceiling mounting is not required and other placements may be used, with the appropriate frequency being used to reduce false positives (i.e., detecting a person that is not within the vehicle, such as a person walking or standing nearby). For example, a plurality of life detection units may be placed underneath or behind one or more seats. In step 208, if no occupant is detected (i.e., received signals are static), the system loops. If occupants are detected (i.e., received signals are not static), an alert will be issued in step 210 to the driver and/or others via wireless technology intended to be received by a smartphone or similar. As an example, a signal may be sent to the owner of the vehicle, first responders, or to a third-party intermediary (e.g., OnStar®). In one embodiment, the vehicle occupant detection system provides for user input, allowing a user to configure the phone numbers and methods of contact. For example, the user input may comprise a keypad, touchscreen interface, wireless connectivity for setup using an application on a smartphone, or any other number of well-known input methods. If a user opens the car door after the first notification issues, the system may terminate. However, if an occupant continues to be detected even after the door opens, the system may continue its processes as if the door was not opened. If a user does not respond (e.g., no one opened a door) and the temperature reaches a second predetermined level at 212, the control unit starts the vehicle and its HVAC system at 214 and a second alert is issued to the driver at 216 and/or other persons to inform them that the vehicle engine has been started. In an alternative embodiment, the second notification to the driver at 216 may occur prior to starting the vehicle, at which point the second notification would alert them that the vehicle will be started within a certain, predetermined time period. It will be appreciated that there are many methods for remotely starting a vehicle that are known in the art. Accordingly, those methods and their safety features are incorporated herein.

While CO monitoring is shown and described, it is not a requirement of the vehicle occupant detection system. Therefore, continuing in FIG. 2, optional CO monitoring begins in step 218 with a CO monitor reading CO levels within the passenger compartment of the vehicle. For example, if the vehicle occupant detection system automatically starts the vehicle, but the vehicle is either in a garage or has a faulty emissions system, the levels of CO can become hazardous. As such, if the CO level approaches (or reaches) a predetermined (dangerous) level in step 220, the vehicle engine is turned off in step 222. If the CO levels are safe, then at step 224 the control unit checks to see if the engine is running (e.g., checking R-terminal on alternator or any other method of determining that the vehicle is running). If the vehicle is either turned off as a result of CO levels in 222 or is unexpectedly shut off (e.g., out of gas), then the windows may be opened partially or fully at 226 and a third alert at 228 may be issued to the driver and/or others notifying them that the windows of the vehicle are partially- or fully-opened. This helps mitigate the risk of CO poisoning while ensuring optimal run time of the HVAC system. At 230, the control unit checks whether the doors have been opened since the control unit initiated. If the doors have still not been opened, the car alarm may be initiated, along with any other urgent alarm system, which may include an audible voice alerting passersby to the occupant in danger. Once the door is opened, the system may terminate. However, in one embodiment, the system will not terminate until 1) the occupant exits the vehicle, or 2) the temperature inside the vehicle is safe.

In one embodiment, a vehicle occupant detection system comprises life detection unit having a microwave transmitter and a microwave receiver. It will be appreciated that the radar components (e.g., microwave transmitter and receiver) may comprise those known in the industry; i.e., a radar system comprises a transmitter producing electromagnetic microwaves, a transmitting antenna, a receiving antenna (often the same antenna is used for transmitting and receiving), and a receiver. The life detection unit(s) may be placed at any number of locations, as discussed previously herein. The transmitter then transmits a microwave signal and the receiver receives the returned signal. The returned signal is then transmitted to a control unit for analysis. The control unit may be in the same physical housing as the life detection unit, or may be separate therefrom. The control unit may comprise a user interface, a microcontroller, a receiver configured to receive the signals transmitted from the life detection unit(s), a means for user input, and a network card (wired, wireless, or equivalent communication protocol, including, Bluetooth, ZigBee, wife, cellular, LoRa, IR, UART, ASK, FSK and others). The means for user input may be a physical keyboard, a touchscreen, voice commands, or wireless connections with a smart device (e.g., smartphone app or similar). While the foregoing radar description is not exhaustive, an exemplary radar system is disclosed in U.S. Patent Application US20140316261A1 titled, “Life Detecting Radars” to Lux et al., which is incorporated herein by reference in its entirety. A signal may be transmitted, using the network card, to alert occupants and third-parties to a triggering event (e.g., occupant detected with temperature exceeding acceptable thresholds, occupant detected while engine is off and doors are locked, etc.), which is accomplished using the microcontroller, based upon logic, to activate the network card and associated signal at the triggering event.

It will be appreciated that other means for detecting an occupant may be used such as audio and visual aids. In other words, the vehicle occupant detection system may also comprise a camera having image recognition capability which can record and identify images within the interior of the vehicle, which is mounted within the interior compartment of the vehicle and is communicatively coupled to the control unit. It may further comprise a sound sensor mounted within the interior compartment of the vehicle and likewise communicatively coupled to the control unit. Further, the occupant detection system may adopt machine learning algorithms such as, but not limited to, convolutional neural network applied to images, sound signals, and CO measurements, to detect the presence of a living occupant inside the vehicle. In one embodiment, to avoid overly-draining the battery, the occupant detection system may sample the environment at defined time intervals, acquiring the necessary information (images, sound, CO levels). In another embodiment, a battery status monitor may be used that will terminate the occupant detection system if the battery gets too low.

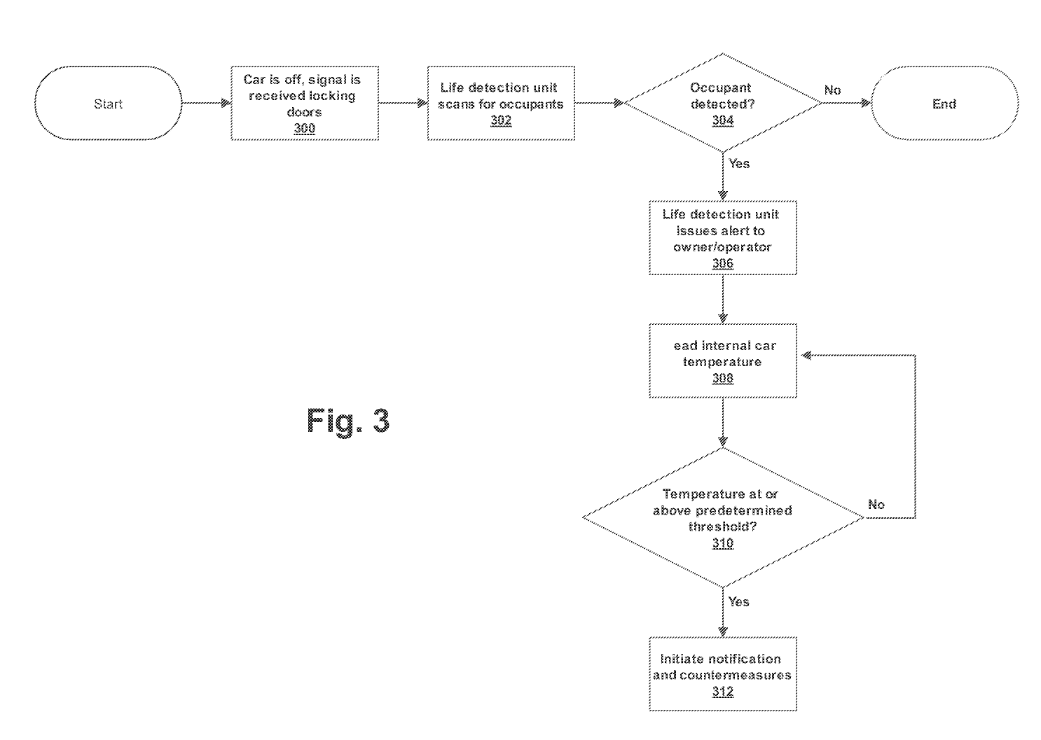

In one embodiment, the vehicle occupant detection system comprises a life detection unit coupled to the computer systems of the vehicle. As shown in FIG. 3, the life detection unit initiates in step 300 once the vehicle is turned off and the doors are locked (such as when a user locks the doors to a vehicle using a key fob). The life detection unit may know that the doors are locked by intercepting the signal from the fob or by monitoring the electrical impulse sent to the doors to lock them. In this embodiment, the life detection unit radar system immediately scans the vehicle for occupants, regardless of temperature. If an occupant is detected, an alert may be issued immediately, which may be an audible alert from the vehicle (e.g., horn honking, alarm, etc.) or may be in the form of a call or message to the owner/operator. By initiating an immediate alert, the risks of harm decreases. Therefore, in step 302, the life detection unit scans the vehicle for occupants (“scans” is understood to mean that the radar components are initiated). The life detection unit then compares the reflected microwave signals with each other to determine if they are static or dynamic (consistent with movement and life). If no life is detected, the system terminates. However, if life is detected, then in step 306 an alert is issued to the owner/operator. The life detection unit then measures the temperature of the passenger compartment of the vehicle, which may be accomplished with an integrated temperature sensor, of, if the life detection unit is mounted outside of the passenger compartment, then using a temperature sensor mounted on in the passenger compartment that is communicatively coupled to the life detection unit. If step 310, if the temperature does not exceed a predetermined threshold, it continues to monitor the temperature. If the temperature is exceeded, then in step 312, additional notifications or countermeasures (as described previously herein) are initiated.

While the examples described above reference starting a vehicle and its accompanying HVAC system, it will be understood that some vehicles may not need to be started to activate the HVAC system. For example, electric cars need not be started for the HVAC system to be activated. Accordingly, such differences are intended to be covered by the scope of this disclosure.

As will be appreciated by the foregoing, the vehicle occupant detection system described herein helps prevent or reduce accidental deaths in a parked vehicle by identifying living occupants (people and animals) using radar systems to detect movement (external or internal) consistent with life, and by providing for mitigating countermeasures to hazardous conditions.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A vehicle occupant detection system, comprising:
a temperature sensor mounted within the passenger compartment of a vehicle;
at least one life detection unit mounted to the vehicle and comprising a microwave transmitter and a microwave receiver, the transmitter configured to transmit a signal within the passenger compartment of the vehicle;
a microcontroller, the microcontroller configured to compare reflected microwave signals to detect movement; and
a network card.

2. The vehicle occupant detection system of claim 1, wherein the microcontroller is configured, based upon preprogrammed logic, to activate the network card when the temperature in the passenger compartment exceeds a predetermined threshold.

3. The vehicle occupant detection system of claim 1, wherein the microcontroller is configured, based upon preprogrammed logic, to activate the network card when an HVAC system of the vehicle is not running, the doors to the vehicle are locked, and an occupant has been detected by the microcontroller comparing microwave signals.

4. The vehicle occupant detection system of claim 1, further comprising a carbon monoxide sensor.

5. The vehicle occupant detection system of claim 1, wherein the microcontroller is configured to start the engine and HVAC system of the vehicle.

6. The vehicle occupant detection system of claim 1, wherein the microcontroller is configured to control the windows and alarm system of the vehicle.

7. A vehicle occupant detection system, comprising:
a control unit coupled to a vehicle;
a temperature sensor mounted within the passenger compartment of the vehicle, the temperature sensor communicatively coupled to the control unit;
a life detection unit configured to detect a heartbeat within the passenger compartment of the vehicle;
a carbon monoxide sensor mounted inside the passenger compartment of the vehicle and communicatively coupled to the control unit;
a camera having image recognition capability which can record and identify images within the interior of the vehicle, mounted within the passenger compartment of the vehicle and communicatively coupled to the control unit; and
a sound sensor mounted within the passenger compartment of the vehicle and communicatively coupled to the control unit.

8. The vehicle occupant detection system of claim 7, wherein the control unit is configured to start the engine and HVAC system of the vehicle.

9. The vehicle occupant detection system of claim 7, wherein the control unit is configured to control the windows and alarm system of the vehicle.

10. A method of using a vehicle occupant detection system to prevent deaths, the method comprising:
using microwave signals within a passenger compartment to detect the presence of a living occupant;
upon determining that the temperature within the passenger compartment is outside of a predetermined range and that an occupant is inside the passenger compartment, notifying one or more individuals that an occupant is inside the passenger compartment and the temperature is outside of the predetermined range.

11. The method of claim 10, wherein the vehicle occupant detection system initiating one or more countermeasures.

12. A vehicle occupant detection system, comprising:
a temperature sensor mounted within a passenger compartment of a vehicle;
a microcontroller having preprogrammed logic, whereupon when the microcontroller reads a temperature from the sensor unit that is outside of a predetermined range, the microcontroller activates a life detection unit;
the life detection unit comprising a microwave transmitter coupled to a vehicle and configured to transmit one or more microwave signals within the passenger compartment of the vehicle, and a microwave receiver configured to receive microwave signals reflected from within the passenger compartment of the vehicle;
wherein the microcontroller is configured to compare the reflected microwave signals one to another; and
upon determining that the reflected microwave signals are dynamic, sending a signal to an owner of the vehicle using a network card, and starting the engine and HVAC system of the vehicle.
